(19) United States
(12) Patent Application Publication
(10) Pub. No.: US 2024/0071624 A1
(43) Pub. Date: Feb. 29, 2024

(54) TECHNIQUES FOR IDENTIFYING POLYCYSTIC OVARY SYNDROME AND ENDOMETRIOSIS FROM WEARABLE-BASED PHYSIOLOGICAL DATA

(71) Applicant: Oura Health Oy, Oulu (FI)

(72) Inventors: Neta A. Gotlieb, Albany, CA (US); Nina Nicole Thigpen, Jersey City, NJ (US)

(21) Appl. No.: 18/454,757

(22) Filed: Aug. 23, 2023

Related U.S. Application Data
(60) Provisional application No. 63/400,458, filed on Aug. 24, 2022.

(51) Int. Cl. G16H 50/30; A61B 5/00; A61B 5/01; A61B 5/024; A61B 5/145; A61B 10/00; G16H 10/60

(52) U.S. Cl. CPC G16H 50/30; A61B 5/01; A61B 5/02405; A61B 5/14542; A61B 5/4806; A61B 5/6802; A61B 5/7275; A61B 5/7435; A61B 10/0012; G16H 10/60; A61B 2010/0019; A61B 2010/0029

(57) ABSTRACT

Methods, systems, and devices for identifying irregular cycles, polycystic ovary syndrome (PCOS), and endometriosis based on wearable-based physiological data are described. A system may be configured to receive physiological data associated with a user collected via a wearable device, the physiological data collected throughout at least a portion of a menstrual cycle for the user. The system may be configured to determine a time series of a plurality of physiological measurements based on the physiological data, and identify that the physiological measurements deviate from a baseline measurements associated with the user, other users, or both. The system may then identify one or more risk metrics associated with relative probabilities that the user is experiencing PCOS, endometriosis, or both, and may generate a message for display on a graphical user interface (GUI) on a user device that indicates information associated with the one or more risk metrics.

### CROSS REFERENCE

The present Application for Patent claims the benefit of U.S. Provisional Patent Application No. 63/400,458 by GOTLIEB et al., entitled “TECHNIQUES FOR IDENTIFYING POLYCYSTIC OVARY SYNDROME AND ENDOMETRIOSIS FROM WEARABLE-BASED PHYSIOLOGICAL DATA,” filed Aug. 24, 2022, assigned to the assignee hereof, and expressly incorporated by reference herein.

### FIELD OF TECHNOLOGY

The following relates to wearable devices and data processing, including techniques for identifying irregular cycles, polycystic ovary syndrome (PCOS), and endometriosis from wearable-based physiological data.

### BACKGROUND

Some wearable devices may be configured to collect data from users. For example, some wearable devices may be configured to detect cycles associated with reproductive health. However, conventional cycle detection techniques implemented by wearable devices are deficient, and may be unable to determine when a user is experiencing reproductive-health related conditions or complications.

### BRIEF DESCRIPTION OF THE DRAWINGS

FIG. 1 illustrates an example of a system that supports techniques for identifying irregular cycles, polycystic ovary syndrome (PCOS), and endometriosis from wearable-based physiological data in accordance with aspects of the present disclosure.

FIG. 2 illustrates an example of a system that supports techniques for identifying irregular cycles, PCOS, and endometriosis from wearable-based physiological data in accordance with aspects of the present disclosure.

FIG. 3 illustrate example menstrual cycle graphs that support techniques for identifying irregular cycles, PCOS, and endometriosis from wearable-based physiological data in accordance with aspects of the present disclosure.

FIG. 4 illustrate example menstrual cycle graphs that support techniques for identifying irregular cycles, PCOS, and endometriosis from wearable-based physiological data in accordance with aspects of the present disclosure.

FIG. 5 illustrates an example of a system that supports techniques for identifying irregular cycles, PCOS, and endometriosis from wearable-based physiological data in accordance with aspects of the present disclosure.

FIG. 6 illustrates an example of a graphical user interface (GUI) that supports techniques for identifying irregular cycles, PCOS, and endometriosis from wearable-based physiological data in accordance with aspects of the present disclosure.

FIG. 7 shows a block diagram of an apparatus that supports techniques for identifying irregular cycles, PCOS, and endometriosis from wearable-based physiological data in accordance with aspects of the present disclosure.

FIG. 8 shows a block diagram of a wearable application that supports techniques for identifying irregular cycles, PCOS, and endometriosis from wearable-based physiological data in accordance with aspects of the present disclosure.

FIG. 9 shows a diagram of a system including a device that supports techniques for identifying irregular cycles, PCOS, and endometriosis from wearable-based physiological data in accordance with aspects of the present disclosure.

FIGS. 10 through 12 show flowcharts illustrating methods that support techniques for identifying irregular cycles, PCOS, and endometriosis from wearable-based physiological data in accordance with aspects of the present disclosure.

### DETAILED DESCRIPTION

Some wearable devices may be configured to collect physiological data from users, including temperature data, heart rate data, and the like. Acquired physiological data may be used to analyze the user's movement and other activities, such as sleeping patterns. Many users have a desire for more insight regarding their physical health, including their sleeping patterns, activity, and overall physical well-being. In particular, many users may have a desire for more insight regarding women's health, including their menstrual cycle, ovulation, fertility patterns, pregnancy, and postpartum period. However, typical cycle tracking or women's health devices and applications lack the ability to provide robust prediction and insight for several reasons.

First, typical cycle prediction applications require users to manually take their temperature with a device at a discrete time each day. This single temperature data point may not provide sufficient context to accurately capture or predict the true temperature variations indicative of woman's health cycle patterns, pregnancy patterns, and postpartum patterns and may be difficult to accurately capture given the sensitivity of the measuring device to user movement or exertion. Second, even for devices that are wearable or that take a user's temperature more frequently throughout the day, typical devices and applications lack the ability to collect other physiological, behavioral, or contextual inputs from the user that can be combined with the measured temperature to more comprehensively understand the complete set of physiological contributors to a woman's cycle, pregnancy, and postpartum period.

Moreover, some users may suffer from conditions that affect their reproductive cycles from an early age. For example, users that experience polycystic ovary syndrome (PCOS) and/or endometriosis may exhibit irregular menstrual and ovarian cycles from a young age. In such cases, the irregular cycles may be “normal” for the user despite the irregular cycles being an adverse side effect of the underlying conditions.

Accordingly, aspects of the present disclosure are directed to techniques for identifying and predicting irregular cycles, PCOS, and endometriosis based on physiological data collected via a wearable device. In particular, computing devices of the present disclosure may receive physiological (e.g., temperature data, heart rate data, heart rate variability (HRV) data, sleep data, blood oxygen data, etc.) from the wearable device associated with the user and determine a time series of physiological measurements taken over multiple days. The physiological data may be collected during one or more stages/phases of a menstrual cycle associated with the user. In this example, the system may compare the time-series of physiological measurements and identify deviations from baseline physiological data collected during a previous menstrual cycle for the user and/or baseline physiological data collected during menstrual cycles of other users. Based on the identified deviations, the system may be configured to identify “risk metrics” associated with relative probabilities or likelihoods that the user is experiencing PCOS and/or endometriosis.

As such, aspects of the present disclosure may detect indications of irregular menstrual/ovulatory patterns that suggest or indicate that the user is suffering from PCOS, endometriosis, or other reproductive health-related conditions. In such cases, an indication of an irregular menstrual/ovarian cycle may be associated with temperature values that deviate from baseline of temperature values of the user collected during prior menstrual cycles. Additionally, or alternatively, the indication of irregular menstrual/ovarian cycles may be identified by comparing the user's physiological data to baseline physiological data associated with menstrual cycles of other users. By comparing the user's data to that of other users, techniques described herein may be able to identify indications of PCOS and/or endometriosis even in cases where the user has exhibited irregular menstrual/ovarian cycles from a young age. In other words, techniques described herein may be able to identify risk metrics for PCOS and endometriosis even in cases where a user is experiencing “normal” or “typical) cycles compared to their own experience, but where the user's cycles would otherwise be considered to be abnormal relative to typical cycles experienced by healthy cycling users.

In some implementations, the system may display information associated with determined risk metrics to the user. In such cases, the user may be able to input additional information or tags that may be used to calculate and/or update identified risk metrics, such as family history information, “tags” for menstruation or pelvic pain, and the like. In such cases, the system may incorporate received user inputs into a predictive function (e.g., a machine learning model for predicting risk metrics associated with reproductive health-related conditions).

In some aspects, techniques described herein may notify a user, clinician, fertility specialist, care-giver, or a combination thereof of the indication of the risk metrics associated with PCOS, endometriosis, and/or other reproductive health-related conditions. For example, a system may generate a message for display on a GUI of a user device that indicates the indication of the one or more risk metrics. In such cases, the system may cause the GUI of a user device to display a message or other notification to notify the user, clinician, etc. of the risk metrics (e.g., relative probabilities that the user is suffering from one of the conditions), make recommendations to the user (e.g., recommendations as to how to reduce a severity of symptoms, recommendation to see a clinician), and the like. In some implementations, the system may make tag recommendations to a user in a personalized manner.

Aspects of the disclosure are initially described in the context of systems supporting physiological data collection from users via wearable devices. Additional aspects of the disclosure are described in the context of example menstrual cycle graphs and an example GUI. Aspects of the disclosure are further illustrated by and described with reference to apparatus diagrams, system diagrams, and flowcharts that relate to techniques for identifying irregular cycles, PCOS, and endometriosis from wearable-based physiological data.

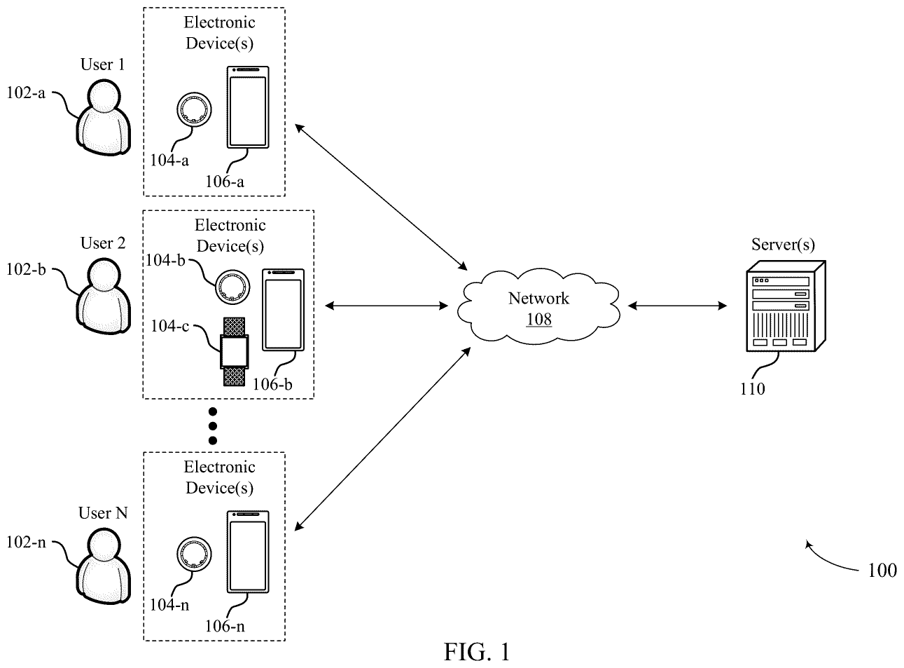

FIG. 1 illustrates an example of a system 100 that supports techniques for identifying irregular cycles, PCOS, and endometriosis from wearable-based physiological data in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, HRV, actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Activity Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for identifying irregular menstrual cycles, PCOS, endometriosis, and other reproductive health-related conditions based on data collected by a wearable device 104. In particular, the system 100 illustrated in FIG. 1 may support techniques for determining risk metrics associated with a relative likelihood or probability that a user 102 is experiencing (or is likely to experience) PCOS, endometriosis, and the like, and causing a user device 106 corresponding to the user 102 to display information associated with the determined risk metrics.

For example, as shown in FIG. 1, User 1 (user 102-a) may be associated with a wearable device 104-a (e.g., ring 104-a) and a user device 106-a. In this example, the ring 104-a may collect data associated with the user 102-a, including temperature, sleep data, heart rate, HRV, respiratory rate, and the like. In particular, the ring 104-a may collect data throughout at least a portion of a menstrual cycle for the user 102-a. In some aspects, data collected by the ring 104-a may be used to determine risk metrics associated with relative probabilities that the user 102-a has experienced, is experiencing, or is likely to experience PCOS, endometriosis, or other reproductive health-related conditions. Identifying risk metrics may be performed by any of the components of the system 100, including the ring 104-a, the user device 106-a associated with User 1, the one or more servers 110, or any combination thereof. Upon identifying the one or more risk metrics, the system 100 may cause a GUI of the user device 106 to display the indication of the one or more risk metrics. In such cases, the user device 106 may be associated with User 1, User 2, User N, or a combination thereof where User 2 and User N may be an example of a clinician, a caregiver, a user associated with User 1, or a combination thereof.

In some implementations, upon receiving physiological data (e.g., including temperature data), the system 100 may determine a time series of physiological measurements taken over a plurality of days of a menstrual cycle. The system 100 may identify that the physiological measurements deviate from baseline physiological data collected from the user 102-a during a prior menstrual cycle, from baseline physiological data associated with menstrual cycles of other users, or both. Additionally, or alternatively, the system 100 may identify that the physiological measurements deviate from a prenatal, perinatal, or postnatal baseline of temperature values for the user. In such cases, the system 100 may identify or calculate the risk metrics associated with PCOS, endometriosis, etc., based on deviations between the user's physiological measurements and the baseline physiological data for the user and/or other users.

In some implementations, the system 100 may generate alerts, messages, or recommendations for User 1, User, 2, and/or User N (e.g., via the ring 104-a, user device 106-a, or both) based on the determined risk metrics, where the messages may provide insights regarding the potential reproductive health-related conditions. In some cases, the messages may provide insight regarding symptoms associated with the one or more reproductive health-related conditions, educational videos and/or text (e.g., content) associated with the one or more reproductive health-related conditions, recommendations to improve symptoms associated with the one or more reproductive health-related conditions, or a combination thereof.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

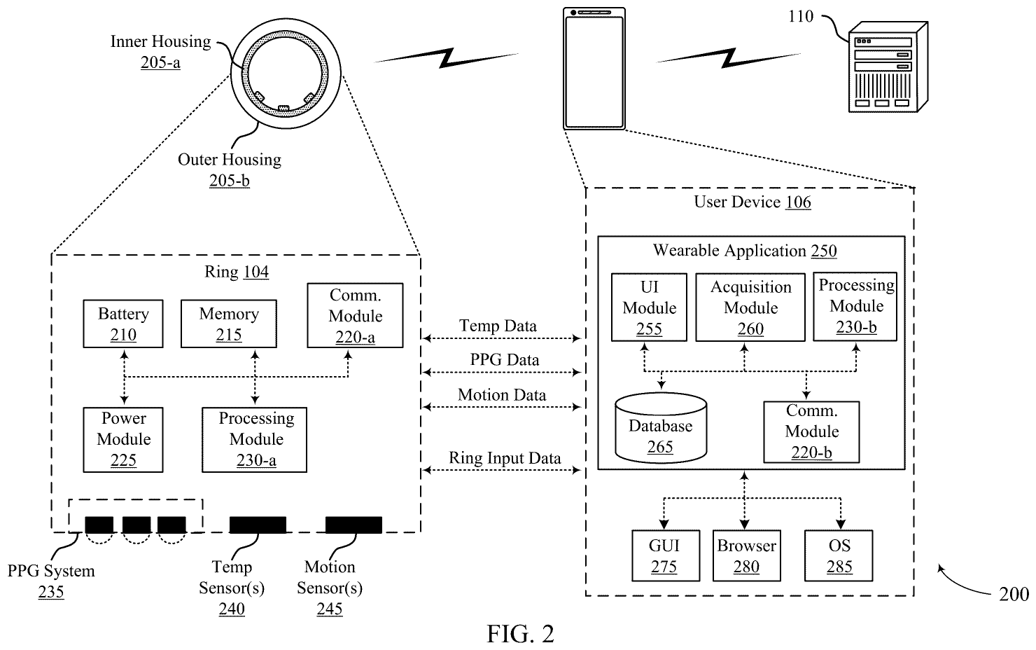

FIG. 2 illustrates an example of a system 200 that supports techniques for identifying irregular cycles, PCOS, and endometriosis from wearable-based physiological data in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the Ibis. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (Meets), and orientation values. Motion counts, regularity values, intensity values, and Meets may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. Meets may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS) 285, a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Activity Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Activity Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Activity Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for identifying irregular menstrual cycles, PCOS, endometriosis, and other reproductive health-related conditions based on data collected by a wearable device 104. In particular, the system 200 illustrated in FIG. 2 may support techniques for determining risk metrics associated with a relative likelihood or probability that a user 102 is experiencing (or is likely to experience) PCOS, endometriosis, and the like, and causing a user device 106 corresponding to the user 102 to display information associated with the determined risk metrics.

Menstrual and ovulatory cycles associated with users are not just associated with the reproductive system, but may be used as a litmus test for the overall health of the user. As such, many physicians consider menstrual/ovulatory cycles as a fifth vital sign for evaluating a user's overall health. Many health conditions may impact the menstrual cycle. As such, wearable devices 104 described herein may be used to tracking menstrual/ovulatory cycles and other cycle-related metrics in order to gain useful insights regarding user's overall health and specific conditions, including reproductive health-related conditions such as PCOS and endometriosis.

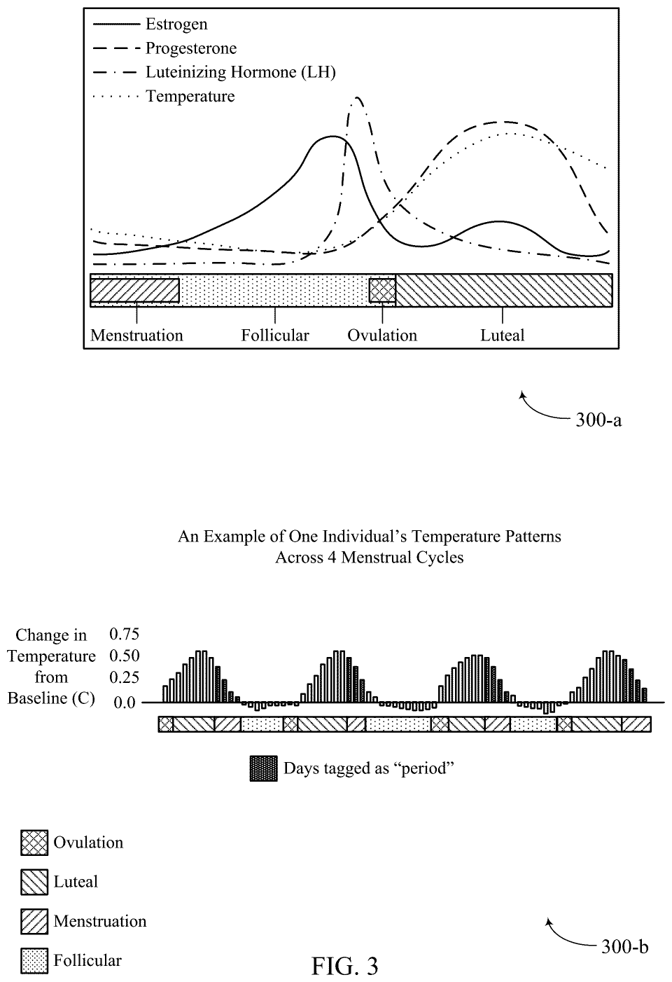

For example, body temperature naturally fluctuates throughout the menstrual cycle, as temperature is highly impacted by hormones such as estrogen and progesterone that fluctuate over the course of the menstrual cycle. Temperature fluctuations throughout a menstrual cycle may be further shown and described with reference to FIG. 3.

FIG. 3 illustrates example menstrual cycle graphs 300-a, 300-b that support techniques for identifying irregular cycles, PCOS, and endometriosis from wearable-based physiological data in accordance with aspects of the present disclosure. Aspects of the menstrual cycle graphs 300-a, 300-b may implement, or be implemented by, aspects of the system 100, the system 200, or both.

The first menstrual cycle graph 300-a illustrates a user's body temperature over the course of the various phases of a menstrual cycle. As shown in the first menstrual cycle graph 300-a, estrogen decreases the user's temperature during the follicular phase (e.g., the first half of the menstrual cycle), where progesterone raises the user's temperature in the luteal phase (e.g., second half of the menstrual cycle).

Further, the second menstrual cycle graph 300-b illustrates a user's temperature patterns across four separate menstrual cycles, where the temperature readings are illustrated relative to the user's baseline temperature. In some aspects, techniques described herein may enable users to “tag” various features or portions of their menstrual cycle to enable the system 200 to more accurately and efficiently identify where the user is within their menstrual cycle. For example, as shown in the second menstrual cycle graph 300-b, a user may be able to “tag” days that they experience a period so that the system 200 may identify where the user is within their menstrual cycle, and to associate acquired physiological data with respective “phases” or stages of their menstrual cycle.

Similar to temperature, other physiological parameters collected via a wearable device 104 (e.g., heart rate, HRV, respiratory rate, oxygen saturation, sleep patterns/data, Sleep Scores, Activity Scores, Readiness Scores) may naturally fluctuate throughout the menstrual cycle. As such, in some implementations, techniques described herein may utilize physiological data and patterns collected via a wearable device 104 throughout a user's menstrual cycles in order to characterize cycles and generate cycle and health-related insights for the user, such as risk metrics associated with certain medical conditions (e.g., PCOS, endometriosis).

Some abnormal menstrual signs that user's should bring to their healthcare providers may include periods lasting longer than ten days, heavy flow that impact the user's daily routine, time between periods being less than 24 days or longer than 38 days, missed periods, frequent spotting and/or bleeding between periods, and the like. However, some users may not experience such symptoms, even in cases where the users are suffering from medical conditions. Accordingly, techniques described herein may leverage physiological data collected via a wearable device 104 to identify health-related conditions associated with the user even in cases where the user may not otherwise exhibit symptoms associated with such conditions.

Some health-related conditions that may be identified by the system 200 may include PCOS and endometriosis. PCOS is a condition that causes irregular menstrual periods due to elevated androgen levels and the fact that monthly ovulation is not occurring. Due to the imbalance of sex hormones with PCOS, eggs may not always mature or be released from the ovary to be fertilized. Instead, the eggs may collect on the ovaries as small, immature follicles, that are mistakenly called cysts (hence the name).

In addition to experiencing long or irregular menstrual cycles and heavy periods, women with PCOS tend to have high baseline levels of luteinizing hormone. As such, ovulation kits may not be reliable for users with PCOS, as such ovulation kits may use a set threshold for positive/negative results. Accordingly techniques described herein may be used to track each user's personal menstrual cycles and pattern based on acquired physiological data in order to learn about ovulation within respective individuals (e.g., how the user's physiological data is changing compared to their own baseline, rather than comparing the user's data to a single data point that is compared to a set threshold that doesn't fit the user).

Approximately five to twenty percent of women of reproductive age suffer from PCOS, making PCOS is one of the most common hormonal endocrine disorders. It has been found that users with PCOS typically have a family history of PCOS. Moreover, people with PCOS may be more susceptible to other health-related conditions as compared to people without PCOS, such as pre-diabetic conditions, diabetes, high cholesterol, and obesity. Accordingly, techniques described herein may enable users to input information such as family history medical data, personal historical medical data, previous diagnoses, and the like, in order to more accurately determine risk metrics for PCOS. People with PCOS can improve their overall health by exercising, eating a nutritious and balanced diet, getting restful sleep, staying hydrated, and taking care of their mental health.

Techniques described herein have been found to identify risk metrics associated with PCOS (e.g., predict PCOS) based on the first day of temperature elevation during the menstrual cycle (relative to the user's own personalized temperature baseline of previous cycles), and the lowest temperature and low temperate duration during the follicular phase, based on the user's personalized follicular baseline. In some aspects, such features (that may be collected via a wearable device) may be input into a machine learning model, such as a binary classifier, to determine a risk metric for PCOS (e.g., whether the user is high risk for PCOS or not). In some cases, the accuracy of calculated risk metrics may be improved by adding cardiac and respiratory features to the machine learning model (in addition to other physiological features).

Another health-related condition that may be identified using techniques described herein is endometriosis. Endometriosis is a chronic inflammatory disease where endometrial tissue (e.g., tissue that normally lines the uterus) grows outside the uterus, such as on the ovaries, behind the uterus, on the bowels, or on the bladder. Rarely, endometrial tissue may grow to other parts of the body. This “misplaced” endometrial tissue can cause pain (mainly pelvic and abdominal), infertility, and very heavy periods.

Approximately ten percent of women throughout the world suffer from endometriosis. However, diagnosis may be challenging, error prone, and invasive. In order to diagnose endometriosis, a clinician may combine a user's medical history, symptoms, clinical exams, and imaging to determine a series of probabilities for various possible medical diagnoses, including endometriosis. Accordingly, aspects of the present disclosure may provide a more reliable, non-invasive method for identifying a relative probability that a user is suffering from endometriosis.

For example, there is an association between the presence of pelvic endometriosis and the appearance of a late decline in body temperature during the early follicular phase of the cycle. Accordingly, by tracking a user's temperature throughout their menstrual cycle and comparing acquired temperature data to the user's own baseline temperature during prior menstrual cycles and/or to baseline temperature data associated with menstrual cycles of other users, techniques described herein may be able to accurately determine a relative likelihood that a user is suffering from endometriosis.

Moreover, women with low resting vagally mediated components of the HRV experience more intense pelvic pain, pain unpleasantness, and a higher number of severe endometriosis-related pain descriptors. Further, women with endometriosis may suffer from higher insomnia severity and lower sleep quality scores (e.g., Sleep Scores) as compared to women without endometriosis. As such, the system 200 may utilize several physiological parameters or measurements acquired by a wearable device 104 in order to evaluate risk scores for endometriosis and other health-related conditions.

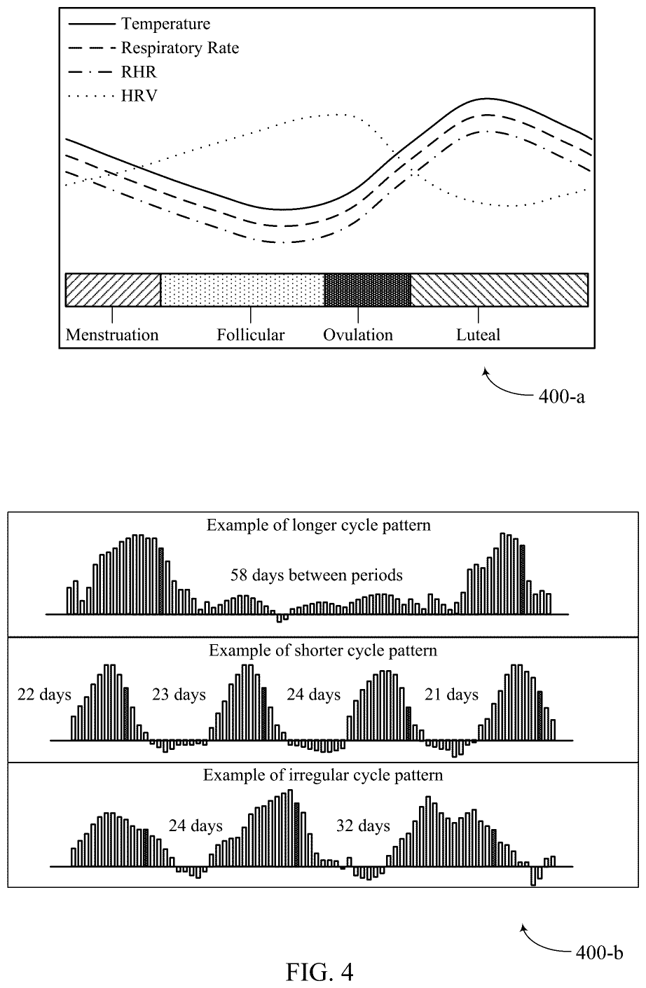

FIG. 4 illustrates example menstrual cycle graphs 400-a, 400-b that support techniques for identifying irregular cycles, PCOS, and endometriosis from wearable-based physiological data in accordance with aspects of the present disclosure. Aspects of the menstrual cycle graphs 400-a, 400-b may implement, or be implemented by, aspects of the system 100, the system 200, the menstrual cycle graphs 300-a, 300-b, or any combination thereof.

In some aspects, the system 200 may be able to accurately track menstrual/ovulatory cycles of varying lengths and detect physiological patterns associated with menstrual/ovulatory cycles. The first menstrual cycle graph 400-a illustrated in FIG. 4 depicts how various physiological parameters/measurements fluctuate throughout a user's menstrual cycle (e.g., fluctuate during different phases of the menstrual cycle). The second menstrual cycle graph 400-b illustrates menstrual/ovulatory cycles for different users that exhibit varying lengths of cycle patterns.

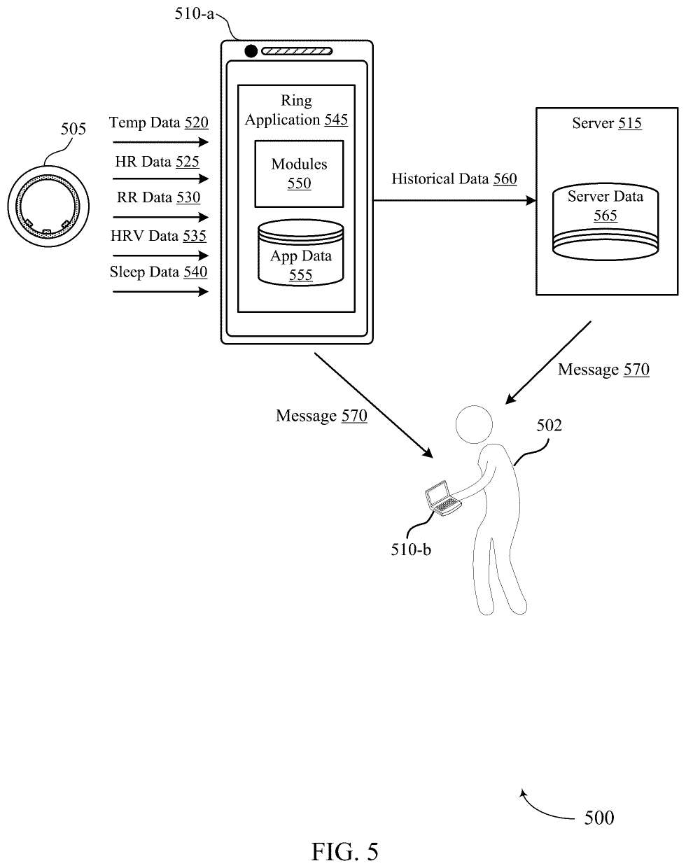

FIG. 5 illustrates an example of a system 500 that supports techniques for identifying irregular cycles, PCOS, and endometriosis from wearable-based physiological data in accordance with aspects of the present disclosure. Aspects of the system 500 may implement, or be implemented by, aspects of the system 100, the system 200, the menstrual cycle graphs 300-a, 300-b, the menstrual cycle graphs 400-a, 400-b, or any combination thereof. In particular, system 500 illustrates an example of a ring 104 (e.g., wearable device 104), a user device 106, and a server 110, as described with reference to FIG. 1.

In some aspects, the system 500 may leverage physiological data (e.g., temperature, heart rate, HRV, respiratory rate, sleep metrics, circadian rhythms, blood oxygen saturation, sleep patterns) collected via a wearable device (e.g., ring 505), user inputs, data from other sources, or any combination thereof, to calculate risk metrics for various health-related conditions (e.g., PCOS and endometriosis) and to provide actionable guidance for the user. User inputs and other information that may be leveraged to determine risk metrics and health-related insights may include, but are not limited to, age, body mass index (BMI), medical history, family history (e.g., historical family medical history), risk factor, and the like. Moreover, user inputs may include “tags” for certain events or subjective feelings, such as tags for pain, (e.g., pelvic pain, abdominal pain), bleeding, heavy menstrual flow, a persistent period, insomnia, etc.

For example, as shown in FIG. 5, the ring 505 may acquire temperature data 520, heart rate data 525, respiratory rate data 530, HRV data 535, sleep data 540, among other forms of physiological data as described herein. In such cases, the ring 505 may transmit temperature data 520, heart rate data 525, respiratory rate data 530, HRV data 535, and sleep data 540 to the user device 510. In some aspects, the data may be acquired throughout one or more menstrual cycles for the user, as described herein. The temperature data 520 may include continuous nighttime temperature data. The respiratory rate data 530 may include continuous nighttime breath rate data. In some cases, multiple devices may acquire physiological data. For example, a first computing device (e.g., user device 510) and a second computing device (e.g., the ring 505) may acquire temperature data 520, heart rate data 525, respiratory rate data 530, HRV data 535, sleep data 540, or a combination thereof.

For example, the ring 505 may acquire user physiological data, such as user temperature data 520, respiratory rate data 530, heart rate data 525, HRV data 535, and sleep data 540, SpO2 data, (e.g., blood oxygen saturation), galvanic skin response, actigraphy, and/or other user physiological data. For example, the ring 505 may acquire raw data and convert the raw data to features with daily granularity. In some implementations, different granularity input data may be used. The ring 505 may send the data to another computing device, such as a mobile device (e.g., user device 510) for further processing.

For example, the user device 510 may identify and/or calculate one or more risk metrics associated with PCOS, endometriosis, or both, based on the received data. In some cases, the system 500 may identify the risk metrics for various health-related conditions based on temperature data 520, respiratory rate data 530, heart rate data 525, HRV data 535, sleep data 540 (e.g., sleep architecture), SpO2 data, galvanic skin response, activity, or a combination thereof. In some cases, the system 500 may determine which features are useful predictors for risk metrics associated with different conditions.

Although the system may be implemented by a ring 505 and a user device 510, any combination of computing devices described herein may implement the features attributed to the system 500. In some cases, the system may smooth the data (e.g., using a 7 day smoothing window or other window). The missing values may be imputed (e.g., using the forecaster Impute method from the python package).

The user device 510 a may include a ring application 545. The ring application 545 may include at least modules 550 and application data 555. In some cases, the application data 555 may include historical temperature patterns for the user and other data. The other data may include temperature data 520, heart rate data 525, respiratory rate data 530, HRV data 535, sleep data 540, or a combination thereof.

The ring application 545 may present information associated with determined/calculated risk metrics (e.g., PCOS risk metric, endometriosis risk metric) to the user. The ring application 545 may include an application data processing module that may perform data processing. For example, the application data processing module may include modules 550 that provide functions attributed to the system 500. Example modules 550 may include a daily temperature determination module, a time series processing module, and a risk metric module.

The daily temperature determination module may determine daily temperature values (e.g., by selecting a representative temperature value for that day from a series of temperature values that were collected continuously throughout the night). The time series processing module may process time series data to identify that the physiological measurements collected throughout a menstrual cycle deviate from baseline physiological data collected from the user throughout prior menstrual cycles of the user.

Additionally, or alternatively, the system may set thresholds that are used to determine risk scores or abnormal patterns associated with the user's physiological data. In such cases, the thresholds may be based on baseline physiological data collected from other, “normal” users during their respective menstrual cycles (e.g., users that are not suffering from PCOS and/or endometriosis). In this regard, in order to calculate risk metrics for the user, the system 500 may be configured to compare acquired physiological data to the user's own baseline physiological data during prior menstrual cycles and/or baseline physiological data collected from other users during their respective menstrual cycles.

Calculated risk scores may be associated with relative probabilities or likelihoods that the user has experienced, is experiencing, or is likely to experience the corresponding medical condition. In this regard, the system 500 may receive user physiological data (e.g., from a ring 505) and output a daily classification of information associated with the calculated risk scores. The ring application 545 may store application data 555, such as acquired temperature data, other physiological data, pregnancy tracking data (e.g., event data), postpartum tracking data (e.g., event data), and risk score metrics.

In some cases, the system 500 may acquire daily user temperature data 520 over an analysis time period. For example, the system 500 may calculate a single temperature value for each day. The system 500 may acquire a plurality of temperature values during the day and/or night and process the acquired temperature values to determine the single daily temperature value. In some implementations, the system 500 may determine a time series of a plurality of temperature values taken over a plurality of days based on the received temperature data 520. The system 500 may identify the risk metrics associated with PCOS and/or endometriosis based on identifying that the user's temperature data deviate from baseline temperature data collected during prior menstrual cycles and/or from baseline temperature data associated with other users.

In some implementations, the system 500 may compute a delta in the time series of the physiological measurements. The delta may be used to calculate risk metrics associated with PCOS, endometriosis, and other health-related conditions. The delta may be representative of a change in one or more hormone levels throughout the user's menstrual cycle.

Similarly, in some cases, the system 500 may determine that one or more other physiological measurements acquired during the user's menstrual cycle deviate from baseline physiological data for corresponding physiological parameters collected during previous menstrual cycles for the user and/or baseline physiological data associated with menstrual cycles for other users. Other physiological parameters that may be evaluated for calculating risk metrics may include, but are not limited to, heart rate data, HRV data, respiratory rate data, blood oxygen saturation data, sleep data (e.g., quantities of sleep disturbances per night), and the like.

In this regard, the system 500 may combine one or more physiological measurements to identify or calculate risk metrics associated with PCOS, endometriosis, and other health-related conditions. In such cases, identifying risk metrics may be based on one physiological measurement or a combination of physiological measurements (e.g., temperature data 520, heart rate data 525, respiratory rate data 530, HRV data 535, sleep data 540). For example, the user's sleep data 540 in combination with the user's temperature data 520 may be an indicator that may the user is suffering from PCOS.

In some cases, the user's logged symptoms (e.g., tags) in combination with the user's physiological data (e.g., temperature data 520, heart rate data 525, respiratory rate data 530, HRV data 535, sleep data 540, or a combination thereof) may be used to calculate risk metrics for PCOS, endometriosis, and other health-related conditions. In such cases, the user's logged symptoms may confirm (e.g., provide a definitive indication of or better prediction of) calculated risk metrics and/or predicted medical conditions. For example, if the system 500 determines that the received temperature data 520 deviates from the user's baseline temperature data and the system receives user input associated indicating abdominal pain or some other symptom, the system may validate or modify determined risk metrics for PCOS and/or endometriosis with greater accuracy and precision than if one of the temperature data 520 deviates from the user's baseline.

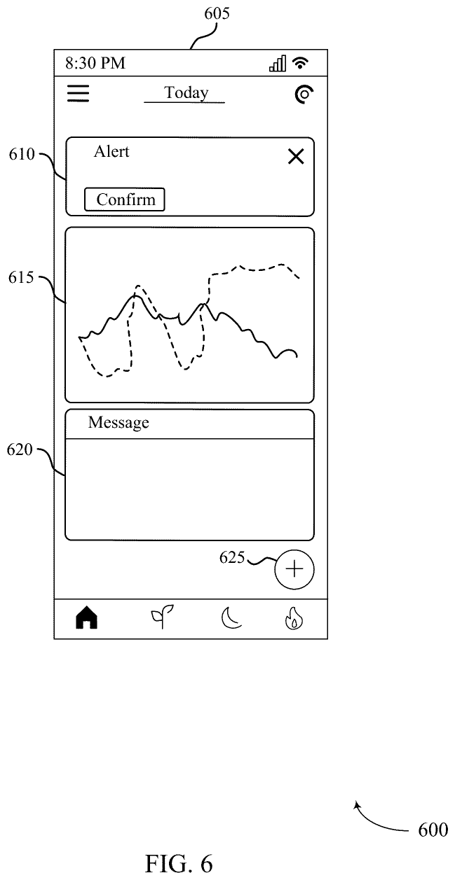

The system 500 may cause a GUI of the user devices 510-a, 510-b to display information associated with determined risk metrics. In some cases, the system 500 may cause the GUI to display the time series. The system 500 may generate a tracking GUI that includes physiological data (e.g., at least temperature data 520), tagged events, and/or other GUI elements described herein with reference to FIG. 6.

The system 500 may generate a number of outputs (e.g., message 570) based on calculated risk metrics. For example, the system 500 may communicate collected physiological data, insights, and/or risk metrics to individual user (e.g., user and/or health care professional), such as via in-app messages. In some cases, the system 500 may communicate the data/insights/risk score to a multi-user platform, such as a health monitoring platform including healthcare providers. For example, a message 570 or a flag may be delivered via a dashboard designed for healthcare providers to keep track of the physiology of their patients. In some cases, the system 500 may provide messages 570 or other alerts when a risk metric for the user exceeds some threshold. In such cases, a healthcare provider and/or the system 500 may reach out to the user for more information, and may refer the user to seek appropriate treatment or diagnosis.

In some cases, the system 500 (e.g., ring application 545) may provide content based on calculated risk metrics, such as videos, podcasts, stories from other users with similar conditions, and the like. The ring application 545 may be configured to provide actionable guidance based on calculated risk metrics, such as actions that the user may take to reduce symptoms associated with identified medical conditions. For example, the ring application 545 may prompt the user to perform structured movements, take a quick walk, perform breathwork exercises, and the like. Similarly, the ring application 545 may provide information about how important sleep is, social support, etc. As the user completes certain exercises or consumes provided materials, the ring application 545 may provide feedback (e.g., in real time) as to how their actions may mitigate or reduce their risk metrics in order to enable the user to take control of their health.

The system 500 may generate a message 570 for display on a GUI on a user device 510-a or 510-b that indicates information associated with identified risk metrics. For example, the system 500 (e.g., user device 510 a or server 515) may transmit the message 570 that indicates the risk metrics associated with one or more medical conditions to the user device 510-b. In such cases, the user device 510-b may be associated with a clinician, a fertility specialist, a care-taker, a partner, or a combination thereof. The detection of a probable medical condition (e.g., PCOS, endometriosis) of may trigger a personalized message 570 to a user highlighting the pattern detected in the user's physiological data and providing an educational link about the respective medical conditions.

In some implementations, the user device 510 may store historical user data. In some cases, the historical user data may include historical data 560. The historical data 560 may include historical temperature patterns of the user, historical heart rate patterns of the user, historical respiratory rate patterns of the user, historical HRV patterns of the user, historical sleep data, historical blood oxygen saturation of the user, historical pregnancy events (e.g., conception date, due date, delivery data, etc.) of the user, historical postpartum events, or a combination thereof. The historical data 560 may be selected from the last few months. The historical data 560 may be used (e.g., by the user device 510 or server 515) to determine a threshold for the user, determine temperature values of the user, predict risk metrics, or a combination thereof. The historical data 560 may be used by the server 515. Using the historical data 560 may allow the user device 510 and/or server 515 to personalize the GUI by taking into consideration the user's historical data 560.

In such cases, the user device 510 may transmit historical data 560 to the server 515. In some cases, the transmitted historical data 560 may be the same historical data stored in the ring application 545. In other examples, the historical data 560 may be different than the historical data stored in the ring application 545. The server 515 may receive the historical data 560. The server 515 may store the historical data 560 in server data 565.

In some implementations, the user device 510 and/or server 515 may also store other data that may be an example of user information. The user information may include, but is not limited to, user age, weight, height, BMI, and gender, and medical history of the user. In some implementations, the user information may be used as features calculating risk metrics associated with medical conditions. The server data 565 may include the other data such as user information.

In some implementations, the system 500 may include one or more user devices 510 for different users. For example, the system 500 may include user device 510-a for a primary user and user device 510-b for a second user 502 associated with the primary user (e.g., partner). The user devices 510 may measure physiological parameters of the different users, provide GUIs for the different users, and receive user input from the different users. In some implementations, the different user devices 510 may acquire physiological information and provide output related to a woman's health, such as menstrual cycles, ovarian cycles, illness, fertility, pregnancy, and/or postpartum. In some implementations, the user device 510-b may acquire physiological information related to the second user 502, such as male illness and fertility.

In some implementations, the system 500 may provide GUIs that inform the second user 502 of relevant information. For example, the first user and the second user 502 may share their information with one another via one or more user devices 510, such as via a server device, mobile device, or other device. In some implementations, the second user 502 may share one or more of their accounts (e.g., usernames, login information, etc.) and/or associated data with one another (e.g., the first user). By sharing information between users, the system 500 may assist second users 502 in making health decisions related to pregnancy. In some implementations, the users may be prompted (e.g., in a GUI) to share specific information. For example, the first user may use a GUI to opt into sharing her pregnancy and/or postpartum information with the second user 502. In such cases, the first user and the second user 502 may receive notifications on their respective user devices 510. In other examples, a second user 502 may make their information (e.g., illness, pregnancy data, postpartum data, etc.) available to the first user via a notification or other sharing arrangement. In such cases, the second user 502 may be an example of a clinician, a fertility specialist, a care-taker, a partner, or a combination thereof.

In some aspects, identified risk metrics may be transmitted to a server or other platform that enables other users (e.g., clinicians, administrators, employers, trainers, etc.) to view the identified risk metrics. For example, the collected data, identified/predicted conditions, and/or generated insights may be transmitted to a multi-user platform or portal so that the user's clinician and other health professionals can make medical treatment decisions based on the identified or predicted conditions.

In some implementations, the system 500 may input physiological data, user inputs, and/or other data sources into a machine learning model (e.g., machine learning model) that is configured to generate risk metrics associated with various medical conditions. In some cases, subsequent diagnoses associated with medical conditions (e.g., positive or negative PCOS diagnoses) may be input into the machine learning model to further train the machine learning model to predict risk metrics.

FIG. 6 illustrates an example of a GUI 600 that supports techniques for identifying irregular cycles, PCOS, and endometriosis from wearable-based physiological data in accordance with aspects of the present disclosure. Aspects of the GUI 600 may implement, or be implemented by, aspects of the system 100, the system 200, the menstrual cycle graphs 300 a, 300 b, the menstrual cycle graphs 400 a, 400 b, the system 500, or any combination thereof. For example, the GUI 600 may be an example of a GUI 275 of a user device 106 (e.g., user device 106 a, 106 b, 106 c) corresponding to a user 102.

In some examples, the GUI 600 illustrates an application page 605 that may be displayed to a user via the GUI 600 (e.g., GUI 275 illustrated in FIG. 2). The server of the system may cause the GUI 600 of the user device (e.g., mobile device) to display inquiries of whether the user activates certain modes associated with reproductive health, such as a fertility mode, a pregnancy mode, a postpartum mode, and the like. In such cases, the system may generate a personalized tracking experience on the GUI 600 of the user device to identify risk metrics for one or more medical conditions such as PCOS and endometriosis.

In some aspects, the application page 605 may display information associated with calculated risk metrics. Information may be displayed via an alert 610, a graph 615, a message 620, or any combination thereof. In some cases, the system may provide content to bring awareness to identified/predicted medical conditions, such as what are the symptoms, what are the risks, and the importance of seeking help and/or treatment. In some cases, the system may deliver personalized messaging based on the birth outcome.

In some cases, the application page 605 may display a prompt to the user to input one or more tags associated identified/predicted medical conditions. For example, the user may be prompted to fill out a questionnaire (e.g., survey). The survey may include indications of symptoms of predicted medical conditions, stress, and/or mood, medical history, family medical history, BMI, age, and the like.

Based on calculated risk metrics and/or input from the user, the system may alert a healthcare provider, provide the user with treatment suggestions and/or referrals to a healthcare provider, provide the user with tools to cope (e.g., take a quick walk, watch this video, complete this breathwork exercise, podcasts, other stores that users can relate to). In some cases, the system may provide stress management techniques (e.g., stress coaching) in response to identified risk metrics.

As shown in FIG. 4, the user may receive an alert 610 associated with identified risk metrics, that may prompt the user to verify or dismiss whether the user has experienced symptoms associated with predicted medical conditions. For example, the system may receive, via the user device and in response to displaying the alert 610, a confirmation that the user has experienced symptoms associated with PCOS and/or endometriosis, such as abdominal pain.

Additionally, in some implementations, the application page 605 may display one or more scores (e.g., Sleep Score, Readiness Score, Activity Score, etc.) for the user for the respective day. Moreover, in some cases, the identified risk metrics may be used to update (e.g., modify) one or more scores associated with the user (e.g., Sleep Score, Activity Score, Readiness Score, etc.). That is, data associated with identified/predicted medical conditions distress may be used to update the scores for the user for the following calendar days. In such cases, the system may notify the user of the score update via alert 610.

In some implementations, the system may be configured to receive user inputs regarding identified/predicted medical conditions (e.g., experienced symptoms, “period” tags, etc.) in order to train classifiers (e.g., supervised learning for a machine learning classifier) and improve risk metric calculations. For example, the user device may receive user inputs 625, and these user inputs 625 may then be input into the classifier to train the classifier. For example, the system may employ a trained a model (e.g., a classifier) to take the last month(s) of a user's data and make a prediction about the probability that a user is suffering from PCOS, endometriosis, and the like. In some implementations, different deep learning representations (e.g., gated recurrent units (GRUs), convolution neural networks (CNNs), LSTMS, Inception Time neural networks, etc.) may be used to derive embeddings that better represent the physiology data for prediction.

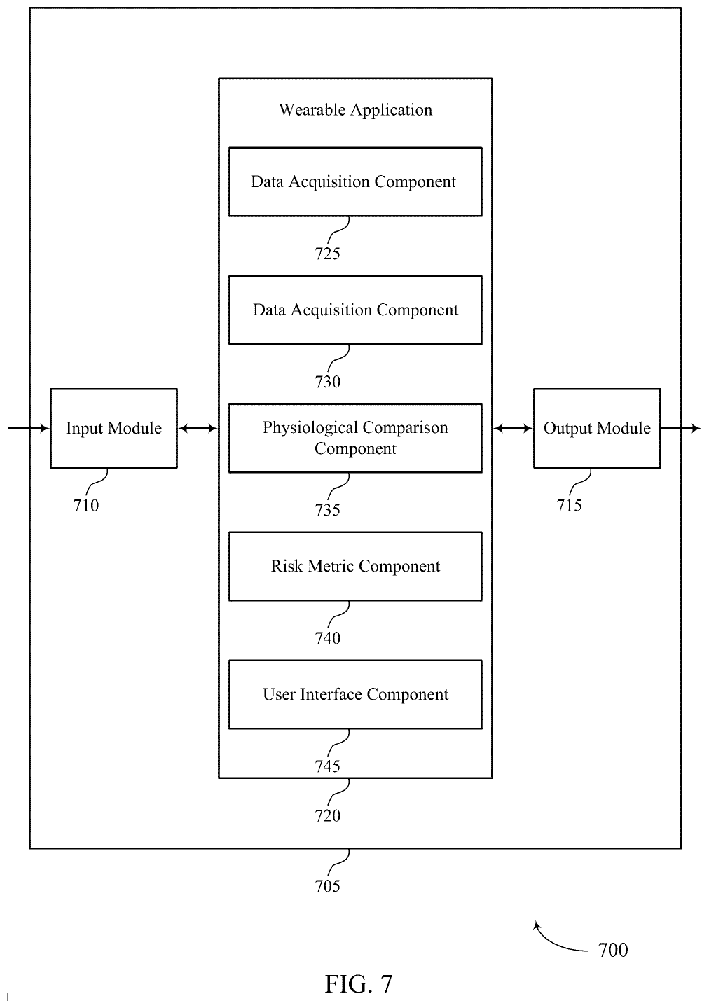

FIG. 7 shows a block diagram 700 of a device 705 that supports techniques for identifying irregular cycles, PCOS, and endometriosis from wearable-based physiological data in accordance with aspects of the present disclosure. The device 705 may include an input module 710, an output module 715, and a wearable application 720. The device 705 may also include a processor. Each of these components may be in communication with one another (e.g., via one or more buses).

The input module 710 may provide a means for receiving information such as packets, user data, control information, or any combination thereof associated with various information channels (e.g., control channels, data channels, information channels related to illness detection techniques). Information may be passed on to other components of the device 705. The input module 710 may utilize a single antenna or a set of multiple antennas.

The output module 715 may provide a means for transmitting signals generated by other components of the device 705. For example, the output module 715 may transmit information such as packets, user data, control information, or any combination thereof associated with various information channels (e.g., control channels, data channels, information channels related to illness detection techniques). In some examples, the output module 715 may be co-located with the input module 710 in a transceiver module. The output module 715 may utilize a single antenna or a set of multiple antennas.

For example, the wearable application 720 may include a data acquisition component 725, a data acquisition component 730, a physiological comparison component 735, a risk metric component 740, a user interface component 745, or any combination thereof. In some examples, the wearable application 720, or various components thereof, may be configured to perform various operations (e.g., receiving, monitoring, transmitting) using or otherwise in cooperation with the input module 710, the output module 715, or both. For example, the wearable application 720 may receive information from the input module 710, send information to the output module 715, or be integrated in combination with the input module 710, the output module 715, or both to receive information, transmit information, or perform various other operations as described herein.

The data acquisition component 725 may be configured as or otherwise support a means for receiving physiological data associated with a user collected via a wearable device, the physiological data collected throughout at least a portion of a menstrual cycle for the user. The data acquisition component 730 may be configured as or otherwise support a means for determining a time series of a plurality of physiological measurements taken over a plurality of days based at least in part on the received physiological data. The physiological comparison component 735 may be configured as or otherwise support a means for identifying that the plurality of physiological measurements deviate from a first set of baseline physiological measurements associated with a previous menstrual cycle for the user, a second set of baseline physiological measurements associated with menstrual cycles for additional users, or both. The risk metric component 740 may be configured as or otherwise support a means for identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both. The user interface component 745 may be configured as or otherwise support a means for generating a message for display on a GUI on a user device that indicates information associated with the one or more risk metrics.

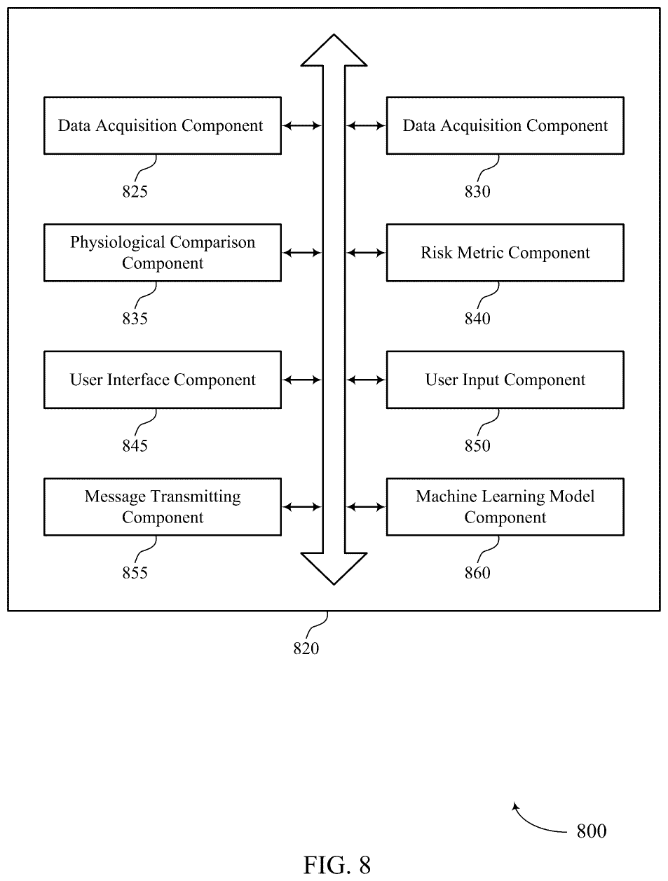

FIG. 8 shows a block diagram 800 of a wearable application 820 that supports techniques for identifying irregular cycles, PCOS, and endometriosis from wearable-based physiological data in accordance with aspects of the present disclosure. The wearable application 820 may be an example of aspects of a wearable application or a wearable application 720, or both, as described herein. The wearable application 820, or various components thereof, may be an example of means for performing various aspects of techniques for identifying irregular cycles, PCOS, and endometriosis from wearable-based physiological data as described herein. For example, the wearable application 820 may include a data acquisition component 825, a data acquisition component 830, a physiological comparison component 835, a risk metric component 840, a user interface component 845, a user input component 850, a message transmitting component 855, a machine learning model component 860, or any combination thereof. Each of these components may communicate, directly or indirectly, with one another (e.g., via one or more buses).

The data acquisition component 825 may be configured as or otherwise support a means for receiving physiological data associated with a user collected via a wearable device, the physiological data collected throughout at least a portion of a menstrual cycle for the user. The data acquisition component 830 may be configured as or otherwise support a means for determining a time series of a plurality of physiological measurements taken over a plurality of days based at least in part on the received physiological data. The physiological comparison component 835 may be configured as or otherwise support a means for identifying that the plurality of physiological measurements deviate from a first set of baseline physiological measurements associated with a previous menstrual cycle for the user, a second set of baseline physiological measurements associated with menstrual cycles for additional users, or both. The risk metric component 840 may be configured as or otherwise support a means for identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both. The user interface component 845 may be configured as or otherwise support a means for generating a message for display on a GUI on a user device that indicates information associated with the one or more risk metrics.

In some examples, the physiological comparison component 835 may be configured as or otherwise support a means for identifying an absence of an ovulatory cycle within the menstrual cycle based at least in part on identifying that the temperature data deviates from baseline temperature data within the first set of baseline physiological measurements, the second set of baseline physiological measurements, or both, wherein identifying the one or more risk metrics, generating the message, or both, is based at least in part on identifying the absence of the ovulatory cycle.

In some examples, the physiological comparison component 835 may be configured as or otherwise support a means for identifying that a portion of the temperature data collected during a follicular phase of the menstrual cycle is lower than baseline follicular phase temperature data associated with the first set of baseline physiological measurements, the second set of baseline physiological measurements, or both, wherein identifying the one or more risk metrics is based at least in part on identifying that the portion of the temperature data is lower than baseline follicular phase temperature data.

In some examples, the physiological comparison component 835 may be configured as or otherwise support a means for computing a delta in the time series of the plurality of physiological measurements based at least in part on determining the time series, wherein identifying that the plurality of physiological measurements deviate from the first set of baseline physiological measurements, the second set of baseline physiological measurements, or both, is based at least in part on computing the delta.

In some examples, the physiological data comprises sleep data, and the physiological comparison component 835 may be configured as or otherwise support a means for determining that a quantity of detected sleep disturbances within received sleep data exceeds a baseline sleep disturbance threshold associated with the previous menstrual cycle for the user for at least a portion of the plurality of days, wherein identifying the one or more risk metrics is based at least in part on determining that the quantity of detected sleep disturbances exceeds the baseline sleep disturbance threshold.

In some examples, the physiological data further comprises HRV data, and the physiological comparison component 835 may be configured as or otherwise support a means for determining that the HRV data is less than a baseline HRV associated with the first set of baseline physiological measurements, the second set of baseline physiological measurements, or both, for at least a portion of the plurality of days, wherein identifying the one or more risk metrics is based at least in part on determining that the HRV data is less than the baseline HRV.

In some examples, the physiological data further comprises heart rate data, and the physiological comparison component 835 may be configured as or otherwise support a means for determining that the heart rate data deviates from a baseline heart rate associated with the first set of baseline physiological measurements, the second set of baseline physiological measurements, or both, for at least a portion of the plurality of days, wherein identifying the one or more risk metrics is based at least in part on determining that the heart rate data deviates from the baseline heart rate.

In some examples, the physiological data further comprises temperature data, and the physiological comparison component 835 may be configured as or otherwise support a means for determining that the temperature data deviates from a baseline temperature associated with the first set of baseline physiological measurements, the second set of baseline physiological measurements, or both, for at least a portion of the plurality of days, wherein identifying the one or more risk metrics is based at least in part on determining that the temperature data deviates from the baseline temperature.

In some examples, the physiological data further comprises blood oxygen saturation data, and the physiological comparison component 835 may be configured as or otherwise support a means for determining that the blood oxygen saturation data deviates from a baseline blood oxygen saturation associated with the first set of baseline physiological measurements, the second set of baseline physiological measurements, or both, for at least a portion of the plurality of days, wherein identifying the one or more risk metrics is based at least in part on determining that the blood oxygen saturation data deviates from the baseline blood oxygen saturation.

In some examples, the user input component 850 may be configured as or otherwise support a means for receiving, via the GUI, a user input indicating an age of the user, a body mass index of the user, a medical history of the user, an indication of birth, an indication of menstruation, one or more tags, one or more surveys, or a combination thereof, wherein identifying the one or more risk metrics is based at least in part on receiving the user input.

In some examples, the message transmitting component 855 may be configured as or otherwise support a means for transmitting the message that indicates information associated with the one or more risk metrics to the user device, wherein the user device is associated with a clinician, the user, or both.

In some examples, the user input component 850 may be configured as or otherwise support a means for receiving, from the user device based at least in part on the message, a user input indicating symptoms, family medical history, or both, associated with PCOS, endometriosis, or both. In some examples, the risk metric component 840 may be configured as or otherwise support a means for updating the one or more risk metrics based at least in part on the user input.

In some examples, the machine learning model component 860 may be configured as or otherwise support a means for inputting the physiological data into a machine learning classifier, wherein identifying the one or more risk metrics is based at least in part on inputting the physiological data into the machine learning classifier.

In some examples, the wearable device comprises a wearable ring device. In some examples, the wearable device collects the physiological data from the user based on arterial blood flow, capillary blood flow, arteriole blood flow, or a combination thereof.

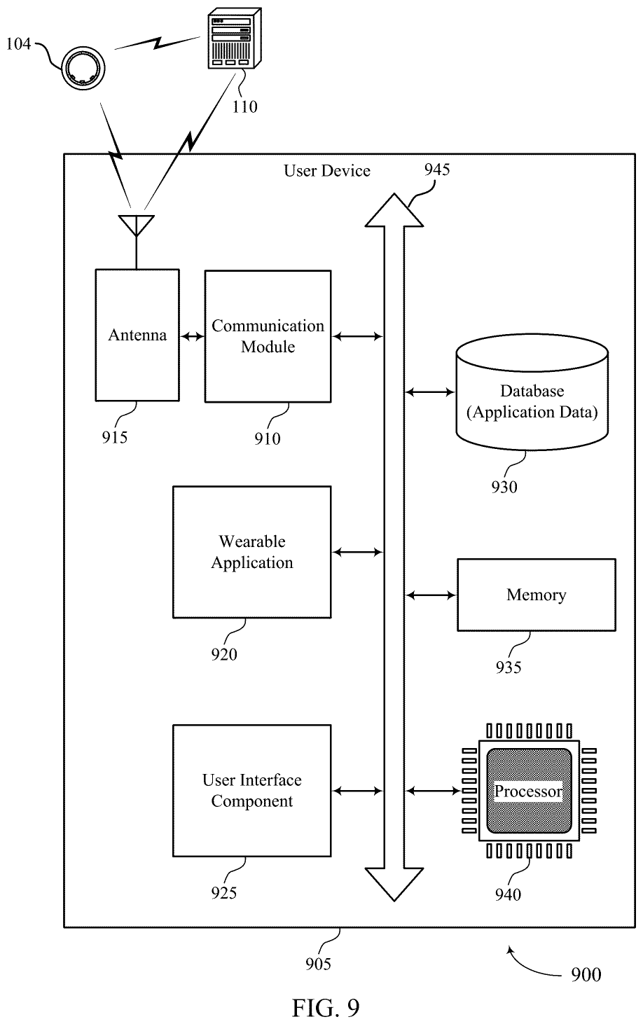

FIG. 9 shows a diagram of a system 900 including a device 905 that supports techniques for identifying irregular cycles, PCOS, and endometriosis from wearable-based physiological data in accordance with aspects of the present disclosure. The device 905 may be an example of or include the components of a device 705 as described herein. The device 905 may include an example of a user device 106, as described previously herein. The device 905 may include components for bi-directional communications including components for transmitting and receiving communications with a wearable device 104 and a server 90, such as a wearable application 920, a communication module 910, an antenna 915, a user interface component 925, a database (application data) 930, a memory 935, and a processor 940. These components may be in electronic communication or otherwise coupled (e.g., operatively, communicatively, functionally, electronically, electrically) via one or more buses (e.g., a bus 945).

The communication module 910 may manage input and output signals for the device 905 via the antenna 915. The communication module 910 may include an example of the communication module 220 b of the user device 106 shown and described in FIG. 2. In this regard, the communication module 910 may manage communications with the ring 104 and the server 90, as illustrated in FIG. 2. The communication module 910 may also manage peripherals not integrated into the device 905. In some cases, the communication module 910 may represent a physical connection or port to an external peripheral. In some cases, the communication module 910 may utilize an operating system such as iOS®, ANDROID®, MS-DOS®, MS-WINDOWS®, OS/2®, UNIX®, LINUX®, or another known operating system. In other cases, the communication module 910 may represent or interact with a wearable device (e.g., ring 104), modem, a keyboard, a mouse, a touchscreen, or a similar device. In some cases, the communication module 910 may be implemented as part of the processor 940. In some examples, a user may interact with the device 905 via the communication module 910, user interface component 925, or via hardware components controlled by the communication module 910.

In some cases, the device 905 may include a single antenna 915. However, in some other cases, the device 905 may have more than one antenna 915, that may be capable of concurrently transmitting or receiving multiple wireless transmissions. The communication module 910 may communicate bi-directionally, via the one or more antennas 915, wired, or wireless links as described herein. For example, the communication module 910 may represent a wireless transceiver and may communicate bi-directionally with another wireless transceiver. The communication module 910 may also include a modem to modulate the packets, to provide the modulated packets to one or more antennas 915 for transmission, and to demodulate packets received from the one or more antennas 915.

The user interface component 925 may manage data storage and processing in a database 930. In some cases, a user may interact with the user interface component 925. In other cases, the user interface component 925 may operate automatically without user interaction. The database 930 may be an example of a single database, a distributed database, multiple distributed databases, a data store, a data lake, or an emergency backup database.

The memory 935 may include RAM and ROM. The memory 935 may store computer-readable, computer-executable software including instructions that, when executed, cause the processor 940 to perform various functions described herein. In some cases, the memory 935 may contain, among other things, a BIOS that may control basic hardware or software operation such as the interaction with peripheral components or devices.

The processor 940 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 940 may be configured to operate a memory array using a memory controller. In other cases, a memory controller may be integrated into the processor 940. The processor 940 may be configured to execute computer-readable instructions stored in a memory 935 to perform various functions (e.g., functions or tasks supporting a method and system for sleep staging algorithms).

For example, the wearable application 920 may be configured as or otherwise support a means for receiving physiological data associated with a user collected via a wearable device, the physiological data collected throughout at least a portion of a menstrual cycle for the user. The wearable application 920 may be configured as or otherwise support a means for determining a time series of a plurality of physiological measurements taken over a plurality of days based at least in part on the received physiological data. The wearable application 920 may be configured as or otherwise support a means for identifying that the plurality of physiological measurements deviate from a first set of baseline physiological measurements associated with a previous menstrual cycle for the user, a second set of baseline physiological measurements associated with menstrual cycles for additional users, or both. The wearable application 920 may be configured as or otherwise support a means for identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both. The wearable application 920 may be configured as or otherwise support a means for generating a message for display on a GUI on a user device that indicates information associated with the one or more risk metrics.

The wearable application 920 may include an application (e.g., “app”), program, software, or other component that is configured to facilitate communications with a ring 104, server 90, other user devices 106, and the like. For example, the wearable application 920 may include an application executable on a user device 106 that is configured to receive data (e.g., physiological data) from a ring 104, perform processing operations on the received data, transmit and receive data with the servers 90, and cause presentation of data to a user 102.

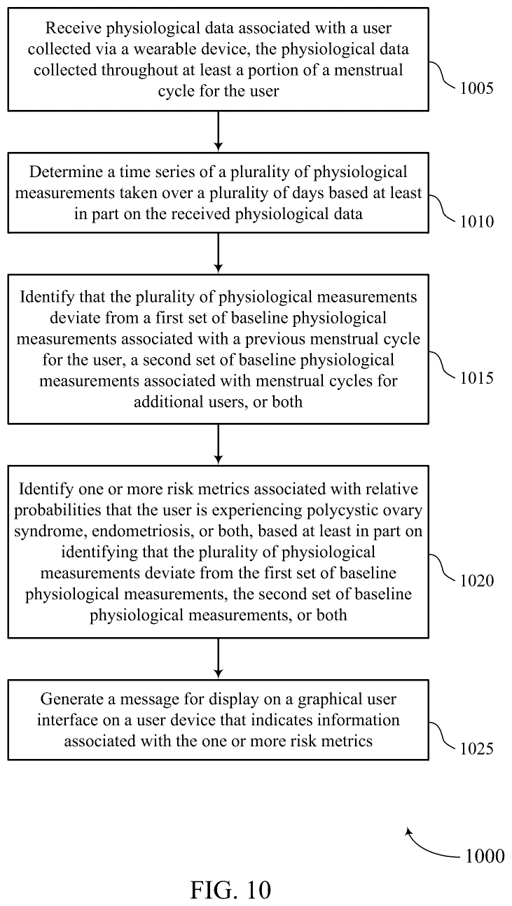

FIG. 10 shows a flowchart illustrating a method 1000 that supports techniques for identifying irregular cycles, PCOS, and endometriosis from wearable-based physiological data in accordance with aspects of the present disclosure. The operations of the method 1000 may be implemented by a user device or its components as described herein. For example, the operations of the method 1000 may be performed by a user device as described with reference to FIGS. 1 through 9. In some examples, a user device may execute a set of instructions to control the functional elements of the user device to perform the described functions. Additionally, or alternatively, the user device may perform aspects of the described functions using special-purpose hardware.

At 1005, the method may include receiving physiological data associated with a user collected via a wearable device, the physiological data collected throughout at least a portion of a menstrual cycle for the user. The operations of 1005 may be performed in accordance with examples as disclosed herein. In some examples, aspects of the operations of 1005 may be performed by a data acquisition component 725 as described with reference to FIG. 7.

At 1010, the method may include determining a time series of a plurality of physiological measurements taken over a plurality of days based at least in part on the received physiological data. The operations of 1010 may be performed in accordance with examples as disclosed herein. In some examples, aspects of the operations of 1010 may be performed by a data acquisition component 730 as described with reference to FIG. 7.

At 1015, the method may include identifying that the plurality of physiological measurements deviate from a first set of baseline physiological measurements associated with a previous menstrual cycle for the user, a second set of baseline physiological measurements associated with menstrual cycles for additional users, or both. The operations of 1015 may be performed in accordance with examples as disclosed herein. In some examples, aspects of the operations of 1015 may be performed by a physiological comparison component 735 as described with reference to FIG. 7.

At 1020, the method may include identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both. The operations of 1020 may be performed in accordance with examples as disclosed herein. In some examples, aspects of the operations of 1020 may be performed by a risk metric component 740 as described with reference to FIG. 7.

At 1025, the method may include generating a message for display on a GUI on a user device that indicates information associated with the one or more risk metrics. The operations of 1025 may be performed in accordance with examples as disclosed herein. In some examples, aspects of the operations of 1025 may be performed by a user interface component 745 as described with reference to FIG. 7.

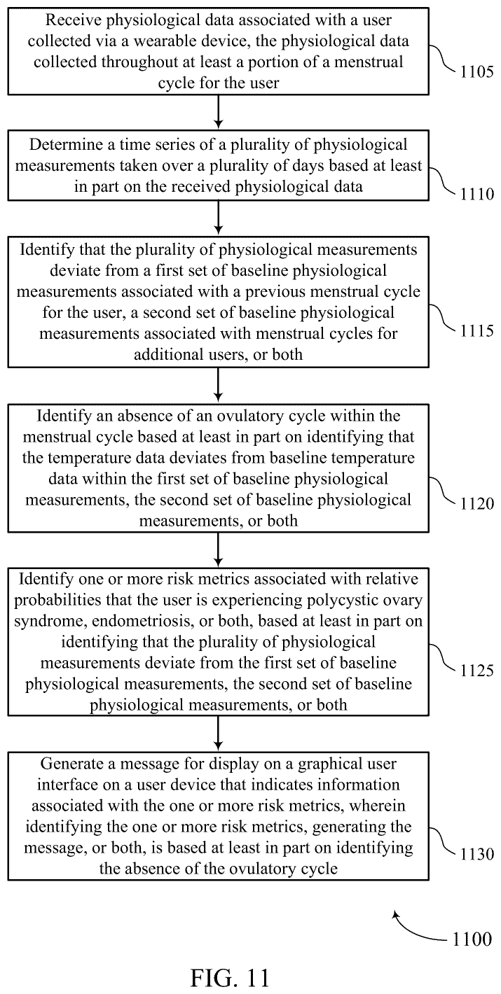

FIG. 11 shows a flowchart illustrating a method 1100 that supports techniques for identifying irregular cycles, PCOS, and endometriosis from wearable-based physiological data in accordance with aspects of the present disclosure. The operations of the method 1100 may be implemented by a user device or its components as described herein. For example, the operations of the method 1100 may be performed by a user device as described with reference to FIGS. 1 through 9. In some examples, a user device may execute a set of instructions to control the functional elements of the user device to perform the described functions. Additionally, or alternatively, the user device may perform aspects of the described functions using special-purpose hardware.

At 1105, the method may include receiving physiological data associated with a user collected via a wearable device, the physiological data collected throughout at least a portion of a menstrual cycle for the user. The operations of 1105 may be performed in accordance with examples as disclosed herein. In some examples, aspects of the operations of 1105 may be performed by a data acquisition component 725 as described with reference to FIG. 7.

At 1110, the method may include determining a time series of a plurality of physiological measurements taken over a plurality of days based at least in part on the received physiological data. The operations of 1110 may be performed in accordance with examples as disclosed herein. In some examples, aspects of the operations of 1110 may be performed by a data acquisition component 730 as described with reference to FIG. 7.

At 1115, the method may include identifying that the plurality of physiological measurements deviate from a first set of baseline physiological measurements associated with a previous menstrual cycle for the user, a second set of baseline physiological measurements associated with menstrual cycles for additional users, or both. The operations of 1115 may be performed in accordance with examples as disclosed herein. In some examples, aspects of the operations of 1115 may be performed by a physiological comparison component 735 as described with reference to FIG. 7.

At 1120, the method may include identifying an absence of an ovulatory cycle within the menstrual cycle based at least in part on identifying that the temperature data deviates from baseline temperature data within the first set of baseline physiological measurements, the second set of baseline physiological measurements, or both. The operations of 1120 may be performed in accordance with examples as disclosed herein. In some examples, aspects of the operations of 1120 may be performed by a physiological comparison component 735 as described with reference to FIG. 7.

At 1125, the method may include identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both. The operations of 1125 may be performed in accordance with examples as disclosed herein. In some examples, aspects of the operations of 1125 may be performed by a risk metric component 740 as described with reference to FIG. 7.

At 1130, the method may include generating a message for display on a GUI on a user device that indicates information associated with the one or more risk metrics, wherein identifying the one or more risk metrics, generating the message, or both, is based at least in part on identifying the absence of the ovulatory cycle. The operations of 1130 may be performed in accordance with examples as disclosed herein. In some examples, aspects of the operations of 1130 may be performed by a user interface component 745 as described with reference to FIG. 7.

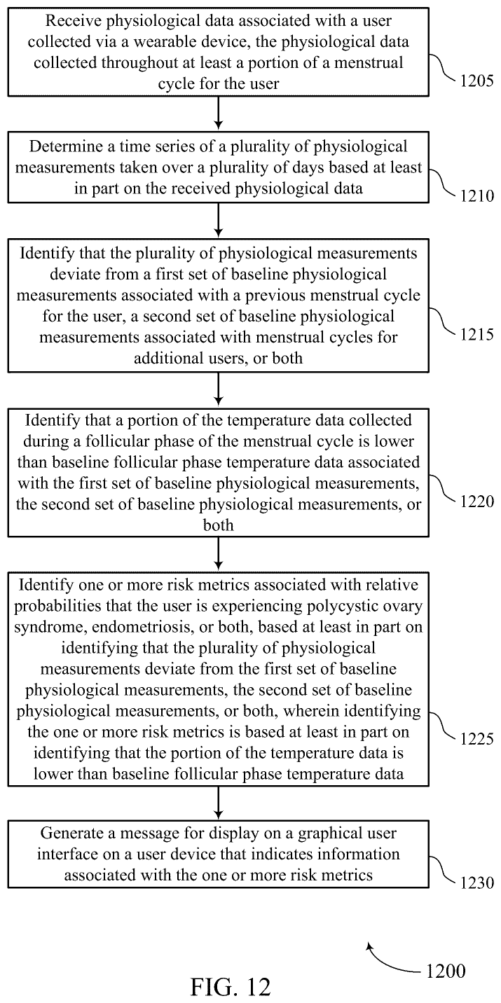

FIG. 12 shows a flowchart illustrating a method 1200 that supports techniques for identifying irregular cycles, PCOS, and endometriosis from wearable-based physiological data in accordance with aspects of the present disclosure. The operations of the method 1200 may be implemented by a user device or its components as described herein. For example, the operations of the method 1200 may be performed by a user device as described with reference to FIGS. 1 through 9. In some examples, a user device may execute a set of instructions to control the functional elements of the user device to perform the described functions. Additionally, or alternatively, the user device may perform aspects of the described functions using special-purpose hardware.

At 1205, the method may include receiving physiological data associated with a user collected via a wearable device, the physiological data collected throughout at least a portion of a menstrual cycle for the user. The operations of 1205 may be performed in accordance with examples as disclosed herein. In some examples, aspects of the operations of 1205 may be performed by a data acquisition component 725 as described with reference to FIG. 7.

At 1210, the method may include determining a time series of a plurality of physiological measurements taken over a plurality of days based at least in part on the received physiological data. The operations of 1210 may be performed in accordance with examples as disclosed herein. In some examples, aspects of the operations of 1210 may be performed by a data acquisition component 730 as described with reference to FIG. 7.

At 1215, the method may include identifying that the plurality of physiological measurements deviate from a first set of baseline physiological measurements associated with a previous menstrual cycle for the user, a second set of baseline physiological measurements associated with menstrual cycles for additional users, or both. The operations of 1215 may be performed in accordance with examples as disclosed herein. In some examples, aspects of the operations of 1215 may be performed by a physiological comparison component 735 as described with reference to FIG. 7.

At 1220, the method may include identifying that a portion of the temperature data collected during a follicular phase of the menstrual cycle is lower than baseline follicular phase temperature data associated with the first set of baseline physiological measurements, the second set of baseline physiological measurements, or both. The operations of 1220 may be performed in accordance with examples as disclosed herein. In some examples, aspects of the operations of 1220 may be performed by a physiological comparison component 735 as described with reference to FIG. 7.

At 1225, the method may include identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both, wherein identifying the one or more risk metrics is based at least in part on identifying that the portion of the temperature data is lower than baseline follicular phase temperature data. The operations of 1225 may be performed in accordance with examples as disclosed herein. In some examples, aspects of the operations of 1225 may be performed by a risk metric component 740 as described with reference to FIG. 7.

At 1230, the method may include generating a message for display on a GUI on a user device that indicates information associated with the one or more risk metrics. The operations of 1230 may be performed in accordance with examples as disclosed herein. In some examples, aspects of the operations of 1230 may be performed by a user interface component 745 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A method is described. The method may include receiving physiological data associated with a user collected via a wearable device, the physiological data collected throughout at least a portion of a menstrual cycle for the user, determining a time series of a plurality of physiological measurements taken over a plurality of days based at least in part on the received physiological data, identifying that the plurality of physiological measurements deviate from a first set of baseline physiological measurements associated with a previous menstrual cycle for the user, a second set of baseline physiological measurements associated with menstrual cycles for additional users, or both, identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both, and generating a message for display on a GUI on a user device that indicates information associated with the one or more risk metrics.

An apparatus is described. The apparatus may include a processor, memory coupled with the processor, and instructions stored in the memory. The instructions may be executable by the processor to cause the apparatus to receive physiological data associated with a user collected via a wearable device, the physiological data collected throughout at least a portion of a menstrual cycle for the user, determine a time series of a plurality of physiological measurements taken over a plurality of days based at least in part on the received physiological data, identify that the plurality of physiological measurements deviate from a first set of baseline physiological measurements associated with a previous menstrual cycle for the user, a second set of baseline physiological measurements associated with menstrual cycles for additional users, or both, identify one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both, and generate a message for display on a GUI on a user device that indicates information associated with the one or more risk metrics.

Another apparatus is described. The apparatus may include means for receiving physiological data associated with a user collected via a wearable device, the physiological data collected throughout at least a portion of a menstrual cycle for the user, means for determining a time series of a plurality of physiological measurements taken over a plurality of days based at least in part on the received physiological data, means for identifying that the plurality of physiological measurements deviate from a first set of baseline physiological measurements associated with a previous menstrual cycle for the user, a second set of baseline physiological measurements associated with menstrual cycles for additional users, or both, means for identifying one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both, and means for generating a message for display on a GUI on a user device that indicates information associated with the one or more risk metrics.

A non-transitory computer-readable medium storing code is described. The code may include instructions executable by a processor to receive physiological data associated with a user collected via a wearable device, the physiological data collected throughout at least a portion of a menstrual cycle for the user, determine a time series of a plurality of physiological measurements taken over a plurality of days based at least in part on the received physiological data, identify that the plurality of physiological measurements deviate from a first set of baseline physiological measurements associated with a previous menstrual cycle for the user, a second set of baseline physiological measurements associated with menstrual cycles for additional users, or both, identify one or more risk metrics associated with relative probabilities that the user is experiencing PCOS, endometriosis, or both, based at least in part on identifying that the plurality of physiological measurements deviate from the first set of baseline physiological measurements, the second set of baseline physiological measurements, or both, and generate a message for display on a GUI on a user device that indicates information associated with the one or more risk metrics.

In some examples of the method, apparatuses, and non-transitory computer-readable medium described herein, and the method, apparatuses, and non-transitory computer-readable medium may include further operations, features, means, or instructions for identifying an absence of an ovulatory cycle within the menstrual cycle based at least in part on identifying that the temperature data deviates from baseline temperature data within the first set of baseline physiological measurements, the second set of baseline physiological measurements, or both, wherein identifying the one or more risk metrics, generating the message, or both, may be based at least in part on identifying the absence of the ovulatory cycle.

In some examples of the method, apparatuses, and non-transitory computer-readable medium described herein, and the method, apparatuses, and non-transitory computer-readable medium may include further operations, features, means, or instructions for identifying that a portion of the temperature data collected during a follicular phase of the menstrual cycle may be lower than baseline follicular phase temperature data associated with the first set of baseline physiological measurements, the second set of baseline physiological measurements, or both, wherein identifying the one or more risk metrics may be based at least in part on identifying that the portion of the temperature data may be lower than baseline follicular phase temperature data.

Some examples of the method, apparatuses, and non-transitory computer-readable medium described herein may further include operations, features, means, or instructions for computing a delta in the time series of the plurality of physiological measurements based at least in part on determining the time series, wherein identifying that the plurality of physiological measurements deviate from the first set of baseline physiological measurements, the second set of baseline physiological measurements, or both, may be based at least in part on computing the delta.

In some examples of the method, apparatuses, and non-transitory computer-readable medium described herein, the physiological data comprises sleep data and the method, apparatuses, and non-transitory computer-readable medium may include further operations, features, means, or instructions for determining that a quantity of detected sleep disturbances within received sleep data exceeds a baseline sleep disturbance threshold associated with the previous menstrual cycle for the user for at least a portion of the plurality of days, wherein identifying the one or more risk metrics may be based at least in part on determining that the quantity of detected sleep disturbances exceeds the baseline sleep disturbance threshold.

In some examples of the method, apparatuses, and non-transitory computer-readable medium described herein, the physiological data further comprises HRV data and the method, apparatuses, and non-transitory computer-readable medium may include further operations, features, means, or instructions for determining that the HRV data may be less than a baseline HRV associated with the first set of baseline physiological measurements, the second set of baseline physiological measurements, or both, for at least a portion of the plurality of days, wherein identifying the one or more risk metrics may be based at least in part on determining that the HRV data may be less than the baseline HRV.

In some examples of the method, apparatuses, and non-transitory computer-readable medium described herein, the physiological data further comprises heart rate data and the method, apparatuses, and non-transitory computer-readable medium may include further operations, features, means, or instructions for determining that the heart rate data deviates from a baseline heart rate associated with the first set of baseline physiological measurements, the second set of baseline physiological measurements, or both, for at least a portion of the plurality of days, wherein identifying the one or more risk metrics may be based at least in part on determining that the heart rate data deviates from the baseline heart rate.

In some examples of the method, apparatuses, and non-transitory computer-readable medium described herein, the physiological data further comprises temperature data and the method, apparatuses, and non-transitory computer-readable medium may include further operations, features, means, or instructions for determining that the temperature data deviates from a baseline temperature associated with the first set of baseline physiological measurements, the second set of baseline physiological measurements, or both, for at least a portion of the plurality of days, wherein identifying the one or more risk metrics may be based at least in part on determining that the temperature data deviates from the baseline temperature.

In some examples of the method, apparatuses, and non-transitory computer-readable medium described herein, the physiological data further comprises blood oxygen saturation data and the method, apparatuses, and non-transitory computer-readable medium may include further operations, features, means, or instructions for determining that the blood oxygen saturation data deviates from a baseline blood oxygen saturation associated with the first set of baseline physiological measurements, the second set of baseline physiological measurements, or both, for at least a portion of the plurality of days, wherein identifying the one or more risk metrics may be based at least in part on determining that the blood oxygen saturation data deviates from the baseline blood oxygen saturation.

Some examples of the method, apparatuses, and non-transitory computer-readable medium described herein may further include operations, features, means, or instructions for receiving, via the GUI, a user input indicating an age of the user, a BMI of the user, a medical history of the user, an indication of birth, an indication of menstruation, one or more tags, one or more surveys, or a combination thereof, wherein identifying the one or more risk metrics may be based at least in part on receiving the user input.

Some examples of the method, apparatuses, and non-transitory computer-readable medium described herein may further include operations, features, means, or instructions for transmitting the message that indicates information associated with the one or more risk metrics to the user device, wherein the user device may be associated with a clinician, the user, or both.

Some examples of the method, apparatuses, and non-transitory computer-readable medium described herein may further include operations, features, means, or instructions for receiving, from the user device based at least in part on the message, a user input indicating symptoms, family medical history, or both, associated with PCOS, endometriosis, or both and updating the one or more risk metrics based at least in part on the user input.

Some examples of the method, apparatuses, and non-transitory computer-readable medium described herein may further include operations, features, means, or instructions for inputting the physiological data into a machine learning classifier, wherein identifying the one or more risk metrics may be based at least in part on inputting the physiological data into the machine learning classifier.

In some examples of the method, apparatuses, and non-transitory computer-readable medium described herein, the wearable device comprises a wearable ring device.

In some examples of the method, apparatuses, and non-transitory computer-readable medium described herein, the wearable device collects the physiological data from the user based on arterial blood flow, capillary blood flow, arteriole blood flow, or a combination thereof.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method comprising:
receiving physiological data associated with a user collected via a wearable device, the physiological data collected throughout at least a portion of a menstrual cycle for the user;
determining a time series of a plurality of physiological measurements taken over a plurality of days based at least in part on the received physiological data;
identifying that the plurality of physiological measurements deviate from a first set of baseline physiological measurements associated with a previous menstrual cycle for the user, a second set of baseline physiological measurements associated with menstrual cycles for additional users, or both;
identifying one or more risk metrics associated with relative probabilities that the user is experiencing polycystic ovary syndrome, endometriosis, or both, based at least in part on identifying that the plurality of physiological measurements deviate from the first set of baseline physiological measurements, the second set of baseline physiological measurements, or both; and
generating a message for display on a graphical user interface on a user device that indicates information associated with the one or more risk metrics.

2. The method of claim 1, wherein the physiological data comprises temperature data, the method further comprising:
identifying an absence of an ovulatory cycle within the menstrual cycle based at least in part on identifying that the temperature data deviates from baseline temperature data within the first set of baseline physiological measurements, the second set of baseline physiological measurements, or both, wherein identifying the one or more risk metrics, generating the message, or both, is based at least in part on identifying the absence of the ovulatory cycle.

3. The method of claim 1, wherein the physiological data comprises temperature data, the method further comprising:
identifying that a portion of the temperature data collected during a follicular phase of the menstrual cycle is lower than baseline follicular phase temperature data associated with the first set of baseline physiological measurements, the second set of baseline physiological measurements, or both, wherein identifying the one or more risk metrics is based at least in part on identifying that the portion of the temperature data is lower than the baseline follicular phase temperature data.

4. The method of claim 1, further comprising:
computing a delta in the time series of the plurality of physiological measurements based at least in part on determining the time series, wherein identifying that the plurality of physiological measurements deviate from the first set of baseline physiological measurements, the second set of baseline physiological measurements, or both, is based at least in part on computing the delta.

5. The method of claim 1, wherein the physiological data comprises sleep data, the method further comprising:
determining that a quantity of detected sleep disturbances within received sleep data exceeds a baseline sleep disturbance threshold associated with the previous menstrual cycle for the user for at least a portion of the plurality of days, wherein identifying the one or more risk metrics is based at least in part on determining that the quantity of detected sleep disturbances exceeds the baseline sleep disturbance threshold.

6. The method of claim 1, wherein the physiological data further comprises heart rate variability data, the method further comprising:
determining that the heart rate variability data is less than a baseline heart rate variability associated with the first set of baseline physiological measurements, the second set of baseline physiological measurements, or both, for at least a portion of the plurality of days, wherein identifying the one or more risk metrics is based at least in part on determining that the heart rate variability data is less than the baseline heart rate variability.

7. The method of claim 1, wherein the physiological data further comprises heart rate data, the method further comprising:
determining that the heart rate data deviates from a baseline heart rate associated with the first set of baseline physiological measurements, the second set of baseline physiological measurements, or both, for at least a portion of the plurality of days, wherein identifying the one or more risk metrics is based at least in part on determining that the heart rate data deviates from the baseline heart rate.

8. The method of claim 1, wherein the physiological data further comprises temperature data, the method further comprising:
determining that the temperature data deviates from a baseline temperature associated with the first set of baseline physiological measurements, the second set of baseline physiological measurements, or both, for at least a portion of the plurality of days, wherein identifying the one or more risk metrics is based at least in part on determining that the temperature data deviates from the baseline temperature.

9. The method of claim 1, wherein the physiological data further comprises blood oxygen saturation data, the method further comprising:
determining that the blood oxygen saturation data deviates from a baseline blood oxygen saturation associated with the first set of baseline physiological measurements, the second set of baseline physiological measurements, or both, for at least a portion of the plurality of days, wherein identifying the one or more risk metrics is based at least in part on determining that the blood oxygen saturation data deviates from the baseline blood oxygen saturation.

10. The method of claim 1, further comprising:
receiving, via the graphical user interface, a user input indicating an age of the user, a body mass index of the user, a medical history of the user, an indication of birth, an indication of menstruation, one or more tags, one or more surveys, or a combination thereof, wherein identifying the one or more risk metrics is based at least in part on receiving the user input.

11. The method of claim 1, further comprising:
transmitting the message that indicates information associated with the one or more risk metrics to the user device, wherein the user device is associated with a clinician, the user, or both.

12. The method of claim 1, further comprising:
receiving, from the user device based at least in part on the message, a user input indicating symptoms, family medical history, or both, associated with polycystic ovary syndrome, endometriosis, or both; and
updating the one or more risk metrics based at least in part on the user input.

13. The method of claim 1, further comprising:
inputting the physiological data into a machine learning classifier, wherein identifying the one or more risk metrics is based at least in part on inputting the physiological data into the machine learning classifier.

14. The method of claim 1, wherein the wearable device comprises a wearable ring device.

15. The method of claim 1, wherein the wearable device collects the physiological data from the user based on arterial blood flow, capillary blood flow, arteriole blood flow, or a combination thereof.

16. An apparatus, comprising:
a processor;
memory coupled with the processor; and
instructions stored in the memory and executable by the processor to cause the apparatus to:
receive physiological data associated with a user collected via a wearable device, the physiological data collected throughout at least a portion of a menstrual cycle for the user;
determine a time series of a plurality of physiological measurements taken over a plurality of days based at least in part on the received physiological data;
identify that the plurality of physiological measurements deviate from a first set of baseline physiological measurements associated with a previous menstrual cycle for the user, a second set of baseline physiological measurements associated with menstrual cycles for additional users, or both;
identify one or more risk metrics associated with relative probabilities that the user is experiencing polycystic ovary syndrome, endometriosis, or both, based at least in part on identifying that the plurality of physiological measurements deviate from the first set of baseline physiological measurements, the second set of baseline physiological measurements, or both; and
generate a message for display on a graphical user interface on a user device that indicates information associated with the one or more risk metrics.

17. The apparatus of claim 16, wherein the physiological data comprises temperature data, and the instructions to are executable by the processor to cause the apparatus to:
identify an absence of an ovulatory cycle within the menstrual cycle based at least in part on identifying that the temperature data deviates from baseline temperature data within the first set of baseline physiological measurements, the second set of baseline physiological measurements, or both, wherein identifying the one or more risk metrics, generating the message, or both, is based at least in part on identifying the absence of the ovulatory cycle.

18. The apparatus of claim 16, wherein the physiological data comprises temperature data, and the instructions to are executable by the processor to cause the apparatus to:
identify that a portion of the temperature data collected during a follicular phase of the menstrual cycle is lower than baseline follicular phase temperature data associated with the first set of baseline physiological measurements, the second set of baseline physiological measurements, or both, wherein identifying the one or more risk metrics is based at least in part on identifying that the portion of the temperature data is lower than the baseline follicular phase temperature data.

19. The apparatus of claim 16, wherein the instructions are further executable by the processor to cause the apparatus to:
compute a delta in the time series of the plurality of physiological measurements based at least in part on determining the time series, wherein identifying that the plurality of physiological measurements deviate from the first set of baseline physiological measurements, the second set of baseline physiological measurements, or both, is based at least in part on computing the delta.

20. The apparatus of claim 16, wherein the physiological data comprises sleep data, and the instructions are further executable by the processor to cause the apparatus to:
determine that a quantity of detected sleep disturbances within received sleep data exceeds a baseline sleep disturbance threshold associated with the previous menstrual cycle for the user for at least a portion of the plurality of days, wherein identifying the one or more risk metrics is based at least in part on determining that the quantity of detected sleep disturbances exceeds the baseline sleep disturbance threshold.

21. The apparatus of claim 16, wherein the physiological data further comprises heart rate variability data, and the instructions are further executable by the processor to cause the apparatus to:
determine that the heart rate variability data is less than a baseline heart rate variability associated with the first set of baseline physiological measurements, the second set of baseline physiological measurements, or both, for at least a portion of the plurality of days, wherein identifying the one or more risk metrics is based at least in part on determining that the heart rate variability data is less than the baseline heart rate variability.

22. The apparatus of claim 16, wherein the physiological data further comprises heart rate data, and the instructions are further executable by the processor to cause the apparatus to:
determine that the heart rate data deviates from a baseline heart rate associated with the first set of baseline physiological measurements, the second set of baseline physiological measurements, or both, for at least a portion of the plurality of days, wherein identifying the one or more risk metrics is based at least in part on determining that the heart rate data deviates from the baseline heart rate.

23. The apparatus of claim 16, wherein the physiological data further comprises temperature data, and the instructions are further executable by the processor to cause the apparatus to:
determine that the temperature data deviates from a baseline temperature associated with the first set of baseline physiological measurements, the second set of baseline physiological measurements, or both, for at least a portion of the plurality of days, wherein identifying the one or more risk metrics is based at least in part on determining that the temperature data deviates from the baseline temperature.

24. The apparatus of claim 16, wherein the physiological data further comprises blood oxygen saturation data, and the instructions are further executable by the processor to cause the apparatus to:
determine that the blood oxygen saturation data deviates from a baseline blood oxygen saturation associated with the first set of baseline physiological measurements, the second set of baseline physiological measurements, or both, for at least a portion of the plurality of days, wherein identifying the one or more risk metrics is based at least in part on determining that the blood oxygen saturation data deviates from the baseline blood oxygen saturation.

25. The apparatus of claim 16, wherein the instructions are further executable by the processor to cause the apparatus to:
receive, via the graphical user interface, a user input indicating an age of the user, a body mass index of the user, a medical history of the user, an indication of birth, an indication of menstruation, one or more tags, one or more surveys, or a combination thereof, wherein identifying the one or more risk metrics is based at least in part on receiving the user input.

26. The apparatus of claim 16, wherein the instructions are further executable by the processor to cause the apparatus to:
transmit the message that indicates information associated with the one or more risk metrics to the user device, wherein the user device is associated with a clinician, the user, or both.

27. The apparatus of claim 16, wherein the instructions are further executable by the processor to cause the apparatus to:
receive, from the user device based at least in part on the message, a user input indicating symptoms, family medical history, or both, associated with polycystic ovary syndrome, endometriosis, or both; and
update the one or more risk metrics based at least in part on the user input.

28. The apparatus of claim 16, wherein the instructions are further executable by the processor to cause the apparatus to:
input the physiological data into a machine learning classifier, wherein identifying the one or more risk metrics is based at least in part on inputting the physiological data into the machine learning classifier.

29. An apparatus, comprising:
means for receiving physiological data associated with a user collected via a wearable device, the physiological data collected throughout at least a portion of a menstrual cycle for the user;
means for determining a time series of a plurality of physiological measurements taken over a plurality of days based at least in part on the received physiological data;
means for identifying that the plurality of physiological measurements deviate from a first set of baseline physiological measurements associated with a previous menstrual cycle for the user, a second set of baseline physiological measurements associated with menstrual cycles for additional users, or both;
means for identifying one or more risk metrics associated with relative probabilities that the user is experiencing polycystic ovary syndrome, endometriosis, or both, based at least in part on identifying that the plurality of physiological measurements deviate from the first set of baseline physiological measurements, the second set of baseline physiological measurements, or both; and
means for generating a message for display on a graphical user interface on a user device that indicates information associated with the one or more risk metrics.

30. A non-transitory computer-readable medium storing code, the code comprising instructions executable by a processor to:
receive physiological data associated with a user collected via a wearable device, the physiological data collected throughout at least a portion of a menstrual cycle for the user;
determine a time series of a plurality of physiological measurements taken over a plurality of days based at least in part on the received physiological data;
identify that the plurality of physiological measurements deviate from a first set of baseline physiological measurements associated with a previous menstrual cycle for the user, a second set of baseline physiological measurements associated with menstrual cycles for additional users, or both;
identify one or more risk metrics associated with relative probabilities that the user is experiencing polycystic ovary syndrome, endometriosis, or both, based at least in part on identifying that the plurality of physiological measurements deviate from the first set of baseline physiological measurements, the second set of baseline physiological measurements, or both; and
generate a message for display on a graphical user interface on a user device that indicates information associated with the one or more risk metrics.
